(19) United States
(12) Patent Application Publication
(10) Pub. No.: US 2005/0112210 A1
(43) Pub. Date: May 26, 2005

(54) Eye nutritional supplement

(76) Inventors: Terry Grossman, Denver, CO (US); Raymond C. Kurzweil, Newton, MA (US)

Correspondence Address: FISH & RICHARDSON PC, 225 FRANKLIN ST, BOSTON, MA, 02110, US

(21) Appl. No.: 10/939,810

(22) Filed: Sep. 13, 2004

Related U.S. Application Data
(60) Provisional application No. 60/502,754, filed on Sep. 12, 2003.

(51) Int. Cl. A61K 35/78; A61K 31/525; A61K 31/51; A61K 31/385; A61K 31/185

(52) U.S. Cl. 424/638; 424/643; 424/655; 424/702; 424/732; 424/752; 424/766; 424/777; 514/440; 514/553; 514/474; 514/763; 514/725; 514/561; 514/562; 514/276; 514/251

(57) ABSTRACT

Nutritional supplements formulated to address eye health are described herein. The supplements include vitamins, minerals, and antioxidants such as vitamins A, C, E, zinc, lutein and zeaxanthin. Also described herein are methods of using nutritional supplements to treat age related macular degeneration.

### CROSS-REFERENCE TO RELATED APPLICATIONS

This application claims priority under 35 USC § 119(e) to U.S. Patent Application Ser. No. 60/502,754, filed on Sep. 12, 2003, the entire contents of which are hereby incorporated by reference.

### TECHNICAL FIELD

This invention relates to nutritional supplements.

### BACKGROUND

Age-related macular degeneration (AMD) is a retinal eye disease that causes progressive loss of central vision, leaving only peripheral or side vision in tact. AMD affects the macula, the central part of the retina responsible for clear, central vision needed for daily activities like reading or driving. As light-sensing cells in the macula called photoreceptors begin to deteriorate, so does the individual's central vision. The extent of vision loss varies widely and is related to the type of AMD, its severity and other individual characteristics; however, it usually does not lead to total blindness.

AMD is the leading cause of severe vision loss (also known as legal blindness) for people over the age of 50 in the Western world. Approximately 25-30 million people are affected by some form of AMD. This number is expected to triple over the next 25 years. Approximately 500,000 new cases of wet AMD are diagnosed annually worldwide. In North America alone, approximately 200,000 new cases of wet AMD are diagnosed each year. It is estimated that 6 million Americans have vision loss from AMD. Based on currently available census data, it is also estimated that another 13-15 million Americans have pre-symptomatic signs of AMD.

The National Eye Institute, a division of the National Institute of Health, recently completed the Age-Related Eye Disease Study (AREDS) to determine if vitamin and mineral supplementation could decrease the risk of AMD. They found that high levels of anti-oxidants and zinc decreased the risk of advanced macular degeneration in high-risk patients by 25%. The researchers who performed this major clinical trial also found that these same nutrients could decrease visual loss by 17%.

### SUMMARY

The particular nutrients in the AREDS Study included: vitamin C, vitamin E, beta-carotene (i.e., vitamin A), and zinc. Very powerful results were found by simply taking these nutrients alone. However, it has been discovered that the combination of the nutrients used in the AREDS study with additional nutrients such as bilberry, lutein and zeaxanthin, provide improved benefits over the nutrients from the AREDS study alone. Accordingly, the administration of antioxidants such as vitamin C, vitamin E, vitamin A (i.e., beta-carotene), and zinc in combination with other naturally occurring antioxidants can provide improved results for the maintenance of eye health.

Moreover, the inventors have discovered that, in some instances, it is desirable to administer lower doses of certain fat soluble nutrients (i.e., vitamins and minerals) than the amounts used in other commercially available nutritional supplements to compensate for the likelihood that a subject could be taking either a multivitamin or could have a diet high in fat soluble nutrients such as beta-carotene or zinc.

In one aspect, the invention features a nutritional supplement including: Vitamin A, Vitamin E, Zinc, Vitamin C, Bilberry, Copper, Selenium, Taurine, Alpha Lipoic Acid, Lutein, Zeaxanthin, Grape Seed Extract, Vincpocetine, Lycopene, and Ginko Biloba.

In some instances, a daily dose of the nutritional supplement includes about 3750-6250 IU Vitamin A, about 300-500 IU Vitamin E, about 30-50 mg Zinc, about 187.5-312.5 mg Vitamin C, about 300-500 mg Bilberry, about 1.5-2.5 mg Copper, 150-250 mcg Selenium, about 300-500 mg Taurine, about 15-25 mg Alpha Lipoic Acid, about 9-15 mg Lutein, about 2.25-3.75 mg Zeaxanthin, about 45-75 mg Grape Seed Extract, about 15-25 mg Vincpocetine, about 22.5-37.5 mg Lycopene, and about 45-75 mg Ginko Biloba, 24%.

In some instances, a daily does of the nutritional supplement includes about 5000 IU Vitamin A, about 400 IU Vitamin E, about 40 mg Zinc, about 250 mg Vitamin C, about 400 mg Bilberry (4:1 Extract), about 2 mg Copper, 200 mcg Selenium, about 400 mg Taurine, about 20 mg Alpha Lipoic Acid, about 12 mg Lutein, about 3 mg Zeaxanthin, about 60 mg Grape Seed Extract, about 20 mg Vincpocetine, about 30 mg Lycopene, and about 60 mg Ginko Biloba, 24%.

In one aspect, the invention features a nutritional supplement including: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

In some instances, a daily dose of the nutritional supplement includes about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

In some instances, a daily dose of the nutritional supplement includes about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

In another aspect, the invention features a nutritional supplement essentially including: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

In some instances, the daily dose of the nutritional supplement essentially includes about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

In some instances, the nutritional supplement essentially includes about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

In another aspect, the invention features a nutritional supplement consisting of: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

In some instances, a daily dose of the nutritional supplement consists of about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

In some instances, a daily dose of the nutritional supplement consists of about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

It is envisioned that each of the nutritional supplements described herein can be in the form of, for example, a capsule or a tablet.

The daily dose of any of the nutritional supplements described herein can be two capsules twice daily.

In another aspect, the invention features a method of treating a subject having age-related macular degeneration. The method includes administering to the patient any one of the nutritional supplements herein described.

In some instances, the method also includes administering to the subject a multivitamin. In other instances, the method also includes administering to the subject an essential fatty acid and docosahexanoic acid.

The term “DHA” refers to docosahexanoic acid. DHA is a fatty acid that has been found to be important in infant eye development.

The term “EFA” refers to essential fatty acids. Essential fatty acids are those fatty acids that are needed for nutritional purposes, but that the body cannot produce itself. Therefore, it is essential that these fatty acids be consumed. Examples of EFAs include Linoleic Acid (LA) and Alpha-Linolenic Acid (LNA).

The details of one or more embodiments of the invention are set forth in the accompanying drawings and the description below. Other features, objects, and advantages of the invention will be apparent from the description and drawings, and from the claims.

### DETAILED DESCRIPTION

The nutritional supplement described herein is designed to provide appropriate amounts of vitamins and minerals to subjects suffering from or at risk of suffering from AMD who possibly take other medications or perhaps other vitamin and mineral supplements. In the cases of fat soluble vitamins and minerals, such as vitamin A, copper and zinc, ingesting too much over a period of time can cause an overdose. Excessive Vitamin A, for example, has been associated with liver toxicity, while too much zinc has been implicated as a risk factor for prostate cancer. Accordingly, the amount of fat soluble vitamins and minerals are featured in lesser amounts than some other vitamin supplements.

The inventors have discovered that of supplements of vitamins, minerals, and natural extracts that have been previously described alone as beneficial, can be administered to a subject in combination, thus providing an effective and convenient supplement for the treatment and/or prevention of eye disorders such as AMD. Some of these ingredients are discussed in detail below.

Vitamins A, C, E, and Zinc

Vitamins A (i.e., beta-carotene), C, E, and Zinc were tested as part of an Age-Related Eye Disease Study, where it was found that subjects treated with a high-dose combination of these vitamins and minerals experienced a reduced risk of vision loss caused by advanced AMD by about 19%. For participants who had either no AMD or early AMD, the supplements did not provide an apparent benefit.

Selenium

Selenium has been implicated in eye health. For example, low levels of Selenium have been implicated in subjects having exudative age-related maculopathy (ARM). For example, in one study, blood samples drawn from 10 exudative ARM patients and 9 healthy-eyed subjects were analyzed, demonstrating that Selenium concentration was significantly lower in the ARM group (186.6 microg/l) than in the control group (207.0 microg/l).

Bilberry

Dry macular degeneration and other age-related eye diseases can be caused by a breakdown of the circulatory system of the eye, and bilberry extracts have been shown to improve microcapillary circulation throughout the body. Additionally, bilberry extracts were used in studies where singlet oxygen was generated in the presence of A2E to reveal that the presence of bilberry, together with other compositions, including vitamin E, butylated hydroxytoluene, resveratrol, and the trolox analogue, reduced A2E-epoxidation by quenching singlet oxygen.

Taurine

Taurine is a conditionally-essential amino acid, which is not utilized in protein synthesis, but rather is found free or in simple peptides. Taurine has been shown to be essential in certain aspects of mammalian development, and in vitro studies in various species have demonstrated that low levels of taurine are associated with various pathological lesions, including cardiomyopathy, retinal degenerations and growth retardation, especially if deficiency occurs during development. Additionally, Taurine is concentrated in the macular region of the retina, where it helps control free radical damage.

Alpha Lipoic Acid

Alpha Lipoic Acid is both a water-soluble and fat-soluble antioxidant, which has been shown to have value in helping to recycle other important antioxidants such as vitamin C, vitamin E and gluatathione.

Lutein and Zeaxanthin

The macular region of the primate retina is yellow in color due to the presence of the macular pigment, composed of two dietary xanthophylls, lutein and zeaxanthin. By absorbing blue-light, the macular pigment protects the underlying photoreceptor cell layer from light damage, possibly initiated by the formation of reactive oxygen species during a photosensitized reaction. There is ample epidemiological evidence that the amount of macular pigment is inversely associated with the incidence of age-related macular degeneration, an irreversible process that is the major cause of blindness in the elderly. The macular pigment can be increased in primates by either increasing the intake of foods that are rich in lutein and zeaxanthin, such as dark-green leafy vegetables, or by supplementation with lutein or zeaxanthin.

Grape Seed Extract

Grape Seed Extract, which is high in Oligomeric Proanthocyanidins (OPC's or PCO's), is powerful antioxidant that can reduce the damage done by free radicals, strengthen and repair connective tissue, and promote enzyme activity. OPC's can also help moderate allergic and inflammatory responses by reducing histamine production

Vinpocetine

Vinpocetine dilates the arteries of the brain but does not dilate other blood vessels. Accordingly, Vinpocetine can improve circulation to the eyes thereby improving vision and improving symptoms occurring in a variety of eyesight disorders.

Lycopene

Lycopene, like lutein and zeaxanthin, are major carotenoids in human blood and tissues. These carotenoids are efficient antioxidants quenching singlet molecular oxygen, which is formed in photooxidative processes and thus may contribute to the prevention of light-exposed tissue, skin and eyes, from light-induced damage.

Gingo Biloba

Senile macular degeneration is a frequent cause of blindness for which there is no satisfactory medical treatment. A double-blind trial comparing Ginkgo biloba extract with a placebo was conducted in 10 out-patients at the Hopital Foch. Drug effectiveness was assessed on the results of fundoscopy and of measurements of visual acuity and visual field. In spite of the small population sample, a statistically significant improvement in long distance visual acuity was observed after treatment with Ginkgo biloba extract.

Other ingredients that have a beneficial effect on the eye include Vitamin B2, Chromium, N-Acetyl-Cysteine, Quercetin, Tumeric, Lycium Berry, Schishandra Berry, and Glycine. Moreover, in cases where Zinc is administered to a subject, is it beneficial to also administer copper.

The methods herein contemplate administration of an effective amount of a composition of compounds to achieve the desired or stated effect (e.g., prevent loss of vision caused by AMD). Typically, the compositions of this invention will be administered from about 1 to about 6 times per day (e.g., 1-3 times per day, or 2 times per day). Such administration can be used as a chronic, acute, or prophylactic therapy. The amount of active ingredient that may be combined with the carrier materials to produce a single dosage form will vary depending upon the patient treated.

A typical preparation will contain about 3750-6250 IU Vitamin A (e.g., about 5000 IU), about 300-500 IU Vitamin E (e.g., about 400 IU), about 2.25-3.75 mg Vitamin B2 (e.g., about 3 mg), about 30-50 mg Zinc (e.g., about 40 mg), about 187.5-312.5 mg Vitamin C (e.g., about 250 mg), about 300-500 mg Bilberry (e.g., about 400 mg), about 1.5-2.5 mg Copper (e.g. about 2 mg), about 150-250 mg Chromium (e.g., about 200), 150-250 mcg Selenium (e.g., about 200 mcg), about 187.5-312.5 mg N-Acetyl-Cysteine (e.g., about 250 mg), about 300-500 mg Taurine (e.g., about 400 mg), about 450-750 mg Quercetin (e.g., about 600 mg), about 15-25 mg Alpha Lipoic Acid (e.g., about 20 mg), about 9-15 mg Lutein (e.g., about 12 mg), about 2.25-3.75 mg Zeaxanthin (e.g., about 3 mg), about 45-75 mg Grape Seed Extract (e.g., about 60 mg), about 45-75 mg Tumeric (e.g., about 60 mg), about 75-125 mg Lycium Berry (e.g., about 100 mg), about 120-200 mg Schishandra Berry (e.g., about 160 mg), about 15-25 mg Vincpocetine (e.g., about 20 mg), about 45-75 mg Glycine (e.g., about 60 mg), about 22.5-37.5 mg Lycopene (e.g., about 30 mg), and about 45-75 mg Ginko Biloba, 24% (e.g., about 60 mg).

The administration of the preparation described above, together with the administration of a multivitamin and a supplement including EPA/DHA marine lipids forms a powerful protective barrier against attacks from environmental toxins and other stresses that are frequently associated with the changes so often seen as part of “normal” aging.

Lower or higher doses than those recited above may be required. Specific dosage and treatment regimens for any particular patient will depend upon a variety of factors, including the activity of the specific compounds employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a composition of this invention can be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

The pharmaceutical compositions of this invention can be orally administered in any acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A number of embodiments of the invention have been described. Nevertheless, it will be understood that various modifications may be made without departing from the spirit and scope of the invention. Accordingly, other embodiments are within the scope of the following claims.

1. A nutritional supplement comprising: Vitamin A, Vitamin E, Zinc, Vitamin C, Bilberry, Copper, Selenium, Taurine, Alpha Lipoic Acid, Lutein, Zeaxanthin, Grape Seed Extract, Vincpocetine, Lycopene, and Ginko Biloba.

2. The nutritional supplement of claim 1, wherein a daily dose of the nutritional supplement comprises about 3750-6250 IU Vitamin A, about 300-500 IU Vitamin E, about 30-50 mg Zinc, about 187.5-312.5 mg Vitamin C, about 300-500 mg Bilberry, about 1.5-2.5 mg Copper, 150-250 mcg Selenium, about 300-500 mg Taurine, about 15-25 mg Alpha Lipoic Acid, about 9-15 mg Lutein, about 2.25-3.75 mg Zeaxanthin, about 45-75 mg Grape Seed Extract, about 15-25 mg Vincpocetine, about 22.5-37.5 mg Lycopene, and about 45-75 mg Ginko Biloba, 24%.

3. The nutritional supplement of claim 1, wherein a daily does of the nutritional supplement comprises about 5000 IU Vitamin A, about 400 IU Vitamin E, about 40 mg Zinc, about 250 mg Vitamin C, about 400 mg Bilberry (4:1 Extract), about 2 mg Copper, 200 mcg Selenium, about 400 mg Taurine, about 20 mg Alpha Lipoic Acid, about 12 mg Lutein, about 3 mg Zeaxanthin, about 60 mg Grape Seed Extract, about 20 mg Vincpocetine, about 30 mg Lycopene, and about 60 mg Ginko Biloba, 24%.

4. The nutritional supplement of claim 1, further comprising at least one of Lycium Berry or Schishandra Berry.

5. The nutritional supplement of claim 1, further comprising Vitamin B2.

6. The nutritional supplement of claim 1, further comprising at least one of glycine or N-acetyl-cystine.

7. The nutritional supplement of claim 1, further comprising chromium.

8. The nutritional supplement of claim 1, further comprising at least one of quercetin or tumeric.

9. A nutritional supplement comprising: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

10. The nutritional supplement of claim 9, wherein a daily dose of the nutritional supplement comprises about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

11. The nutritional supplement of claim 9, wherein a daily does of the nutritional supplement comprises about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

12. A nutritional supplement consisting essentially of: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

13. The nutritional supplement of claim 12, wherein a daily dose of the nutritional supplement consists essentially of about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

14. The nutritional supplement of claim 12, wherein a daily does of the nutritional supplement consists essentially of about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

15. The nutritional supplement of claim 1, further comprising a carrier.

16. The nutritional supplement of claim 15, wherein the carrier is lactose or corn starch.

17. A nutritional supplement consisting of: Vitamin A, Vitamin E, Vitamin B2, Zinc, Vitamin C, Bilberry, Copper, Chromium, Selenium, N-Acetyl-Cysteine, Taurine, Quercetin, Alpha Lipoic Acid, Lutein, Zeaxanthin, Grape Seed Extract, Tumeric, Lycium Berry, Schishandra Berry, Vincpocetine, Glycine, Lycopene, and Ginko Biloba.

18. The nutritional supplement of claim 17, wherein a daily dose of the nutritional supplement consists of about 3750-6250 IU Vitamin A, about 300-500 IU Vitamin E, about 2.25-3.75 mg Vitamin B2, about 30-50 mg Zinc, about 187.5-312.5 mg Vitamin C, about 300-500 mg Bilberry, about 1.5-2.5 mg Copper, about 150-250 mg Chromium, 150-250 mcg Selenium, about 187.5-312.5 mg N-Acetyl-Cysteine, about 300-500 mg Taurine, about 450-750 mg Quercetin, about 15-25 mg Alpha Lipoic Acid, about 9-15 mg Lutein, about 2.25-3.75 mg Zeaxanthin, about 45-75 mg Grape Seed Extract, about 45-75 mg Tumeric, about 75-125 mg Lycium Berry, about 120-200 mg Schishandra Berry, about 15-25 mg Vincpocetine, about 45-75 mg Glycine, about 22.5-37.5 mg Lycopene, and about 45-75 mg Ginko Biloba, 24%.

19. The nutritional supplement of one of claims 17, wherein a daily does of the nutritional supplement consists of about 5000 IU Vitamin A, about 400 IU Vitamin E, about 3 mg Vitamin B2, about 40 mg Zinc, about 250 mg Vitamin C, about 400 mg Bilberry (4:1 Extract), about 2 mg Copper, about 200 mg Chromium, 200 mcg Selenium, about 250 mg N-Acetyl-Cysteine, about 400 mg Taurine, about 600 mg Quercetin, about 20 mg Alpha Lipoic Acid, about 12 mg Lutein, about 3 mg Zeaxanthin, about 60 mg Grape Seed Extract, about 60 mg Tumeric, about 100 mg Lycium Berry, about 160 mg Schishandra Berry, about 20 mg Vincpocetine, about 60 mg Glycine, about 30 mg Lycopene, and about 60 mg Ginko Biloba, 24%.

20. The nutritional supplement of claim 1, wherein the nutritional supplement is in the form of a capsule.

21. The nutritional supplement of claim 1, wherein the nutritional supplement is in the form of a tablet.

22. The nutritional supplement of claim 1, wherein the daily dose consists of two capsules twice daily.

23. A method of treating a subject having age-related macular degeneration, the method comprising administering to the patient any one of the nutritional supplement of claim 1.

24. The method of claim 23, further comprising administering to the subject a multivitamin.

25. The method of claim 23, further comprising administering to the subject an essential fatty acid and docosahexanoic acid.
